(19) United States
(12) Patent Application Publication
(10) Pub. No.: US 2012/0100602 A1
(43) Pub. Date: Apr. 26, 2012

(54) BIOREACTOR SYSTEM FOR MECHANICAL STIMULATION OF BIOLOGICAL SAMPLES

(76) Inventors: Helen H Lu, New York, NY (US); Mario Castillo, Cincinati, OH (US); Brooth R. Dargis, New York, NY (US)

(21) Appl. No.: 13/131,574

(22) PCT Filed: Dec. 8, 2009
(86) PCT No.: PCT/US09/06453

§ 371 Date: Jan. 10, 2012

Related U.S. Application Data
(60) Provisional application No. 61/120,580, filed on Dec. 8, 2008.

(51) Int. Cl. C12M 1/00

(52) U.S. Cl. 435/289.1

(57) ABSTRACT

The subject application provides an apparatus for applying mechanical loading to a biological sample, said apparatus comprising: a bioreactor device configured to house the biological sample, and a load application unit for applying mechanical loading to the biological sample housed in the bioreactor device, said load application unit including: a) a first sliding arm for applying mechanical displacement to the biological sample in the bioreactor device in a first predetermined direction; and b) a guide rail coupled to the first sliding arm to limit movement of the first sliding arm such that the first sliding arm moves only in the first predetermined direction when displaced.

This application claims the benefit of U.S. Provisional Application No. 61/120,580, filed Dec. 8, 2008, the entire contents of which are hereby incorporated by reference herein.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND

Mechanical loading has been shown to aid in proper differentiation of a number of different types of cells and corresponding tissue to a state closer to the native cells and tissues which must withstand a variety of types of mechanical loading (tensile, compressive, etc.) to function in vivo.

A system is desired in which cells, scaffolds or tissue (engineered or graft) can be loaded and grown while being subject to a variety of mechanical loads. Such a system can aid in the testing and understanding of the mechanical and growth conditions necessary for creating properly conditioned tissue grafts and properly differentiated tissues.

### BRIEF SUMMARY

The subject application provides an apparatus for applying mechanical loading to a biological sample, said apparatus comprising: a bioreactor device configured to house the biological sample, and a load application unit for applying mechanical loading to the biological sample housed in the bioreactor device, said load application unit including: a) a first sliding arm for applying mechanical displacement to the biological sample in the bioreactor device in a first predetermined direction; and b) a guide rail coupled to the first sliding arm to limit movement of the first sliding arm such that the first sliding arm moves only in the first predetermined direction when displaced.

Other inventive aspects and features are discussed in the following detailed description.

### BRIEF DESCRIPTION OF THE FIGURES

FIG. 1: FIG. 1A is a schematic showing plan and isometric views of one embodiment of the bioreactor device.

The device acts as a bioreactor by employing a cell-friendly environment with the use of FDA-compliant material.

FIG. 1B is a top view photograph of the bioreactor device with feeding line used to change media, without exposing the system to ambient environment.

FIG. 2: FIG. 2A shows an enlarged schematic view of the bioreactor device shown in FIG. 1A.

FIG. 2B is an exploded view of the bioreactor device shown in FIG. 1A.

FIG. 3: FIG. 3A is a schematic showing plan and isometric views of a load application unit. The load application unit provides a stable platform for the necessary mechanical loads and smooth movement.

FIG. 3B is an isometric photograph of a load application unit with flexible coupling during an initial stage dry run.

FIG. 4: FIG. 4A shows the load application unit plate.

FIG. 4B shows the load application unit sliding arm.

FIG. 4C shows the load application unit guide rail spacer.

FIG. 5: FIG. 5A shows a schematic view of Device 2i.1.

FIG. 5B shows the exploded view of Device 2i.1.

FIG. 6: FIG. 6A shows a schematic view of Device 2i.2.

FIG. 6B shows the exploded view of Device 2i.2.

FIG. 7: FIG. 7A shows a schematic view of Device 2i.3.

FIG. 7B shows the exploded view of Device 2i.3.

FIG. 8: FIG. 8A shows a schematic view of Device 2i.4.

FIG. 8B shows the exploded view of Device 2i.4.

FIG. 9: FIG. 9A shows a schematic view of Device 2i.5.

FIG. 9B shows the exploded view of Device 2i.5.

FIG. 10: FIG. 10 shows one exemplary embodiment of the present invention. (SLS coupled to four Bioreactor Devices)

FIG. 11: FIG. 11 shows the schematic of one embodiment of the apparatus 108 according to the present invention. In this embodiment, one set of guiding rails 101 guides the linear displacement of sliding arm 103 which is actuated by actuator 105. A second set of guiding rails 102 guides the linear displacement of sliding arm 104 which is actuated by actuator 106. The two sliding arms apply mechanical loading to bioreactor devices 107 from two different directions.

FIG. 12: FIG. 12A shows the CAD model drawing of one exemplary embodiment of the Bioreactor Device base.

FIG. 12B shows the photograph of the exemplary embodiment of the Bioreactor Device base shown in FIG. 12A.

FIG. 13: FIG. 13A shows the side view of one embodiment of the Bioreactor Device according to the present invention.

FIG. 13B shows the side view of one preferred embodiment of the Bioreactor Device according to the present invention.

FIG. 14: FIG. 14A shows the drawing of one preferred embodiment of the Bioreactor Device cover according to the present invention. The Device cover shown includes a slot for guiding the movement of the insertion piece.

FIG. 14B shows the exploded view of the Bioreactor Device cover shown in FIG. 14B.

FIG. 14C shows the drawing of another embodiment of the Bioreactor Device cover according to the present invention.

FIG. 15: FIG. 15A shows the drawing of one embodiment of the SLS Plate design according to the present invention.

FIG. 15B shows the drawing of one preferred embodiment of the SLS Plate design according to the present invention.

FIG. 16: FIG. 16A shows the CAD model drawing of one exemplary embodiment of the Bioreactor Device.

FIG. 16B shows the photograph of the exemplary embodiment of the Bioreactor Device shown in FIG. 16A.

### DETAILED DESCRIPTION

### Terms

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “actuator” means a device for moving or controlling another device or set of devices by converting energy from another source, e.g., a motor, into mechanical motion.

As used herein, “bioreactor device” means, in its broadest sense, any device or system that supports a biologically active environment.

As used herein, “control system” means a device or set of devices which regulates the behavior of other devices. In one embodiment, it can be a computer which automatically or with user interface controls other devices via electronic signals or wireless signals.

As used herein, “design dimension” shall mean the desired dimension to achieve during raw material machining and is determined by the dimensions in the design.

As used herein, “functional” shall mean affecting physiological or psychological functions but not organic structure.

As used herein, “graft” shall mean, in its broadest sense, any device to be implanted during a surgical procedure to transplant tissue without a blood supply, including but not limited to soft tissue graft, synthetic grafts, and the like. The graft can be an allograft, for example, tissue taken from one person for transplantation into another, or an autograft.

An “allograft” is tissue taken from one person for transplantation into another. An “autograft” or “autologous graft” is a graft comprising tissue taken from the same subject to receive the graft. Graft can also be allogeneic or xenogenic. As used herein, “allogeneic” means from the same species. As applied to a graft, allogeneic means that the graft is derived from a material originating from the same species as the subject receiving the graft. “Xenogenic” means from a different species. As applied to grafts, xenogenic shall mean that the graft is derived from a material originating from a species other than that of the subject receiving the graft.

As used herein, “load application unit” means, in its broadest sense, any device or system that applies mechanical loading including, for example, compression, tension, torque, etc.

As used herein, “mechanical loading” shall mean forces applied to a structure or a component which are mechanical in nature, or a mechanical force. Mechanical loading includes, for example, compression, tension, torque, etc.

As used herein, “polymer” shall mean, in its broadest sense, a chemical compound or mixture of compounds formed by polymerization and including repeating structural units. Polymers may be constructed in multiple forms and compositions or combinations of compositions.

As used herein, all numerical ranges provided are intended to expressly include at least the endpoints and all numbers that fall between the endpoints of ranges.

### EMBODIMENTS

In an aspect of this disclosure, an apparatus for applying mechanical loading to a biological sample is provided, which apparatus comprises a bioreactor device configured to house the biological sample, and a load application unit for applying mechanical loading to the biological sample housed in the bioreactor device, said load application unit including: a) a first sliding arm for applying mechanical displacement to the biological sample in the bioreactor device in a first predetermined direction; and b) a guide rail coupled to the first sliding arm to limit movement of the first sliding arm such that the first sliding arm moves only in the first predetermined direction when displaced.

The above-mentioned apparatus can include various features. For example, in an exemplary embodiment, the bioreactor device comprises: c) a first securing device for securing one end of the biological sample; and d) a second securing device for securing another end of the biological sample opposite to the first end. In another exemplary embodiment, the load application unit further comprises an insert piece to couple the first sliding arm to the first securing device or the second securing device.

In addition, the load application unit can be configured to apply mechanical loading in any one or a combination of directions to the biological sample in the bioreactor device. The mechanical loading can be compression, tension, a hybrid, etc. In one exemplary embodiment, the first predetermined direction in which the sliding arm is displaced is perpendicular to a longitudinal axis of the insert piece. In another exemplary embodiment, the first predetermined direction in which the sliding arm is displaced is perpendicular to a longitudinal axis of the sliding arm.

In another exemplary embodiment, the bioreactor device further comprise clamps, specifically, the bioreactor device can further comprise f) a static clamp for securing one end of the biological sample; and g) a sliding clamp for securing another end of the biological sample opposite to the first end, said sliding clamp being configured for movement in said first predetermined direction. The clamp can be constructed from a polymer or a mixture of polymers which can include, for example, polytetrafluoroethylene (PTFE).

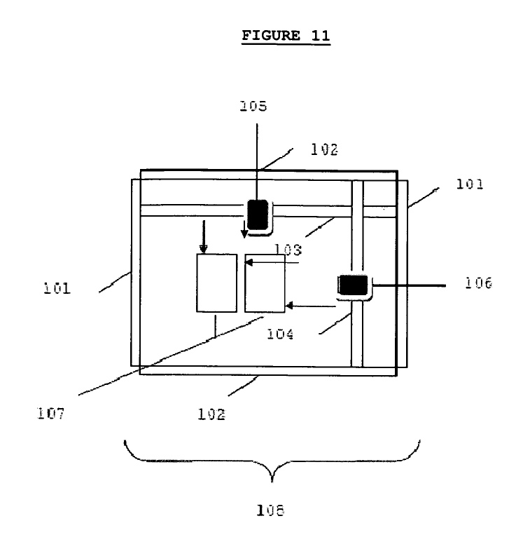

In an exemplary embodiment, the load application unit further comprises a first actuator connected to the first sliding arm for actuating the mechanical displacement of the sliding arm in the first predetermined direction. The load application unit further can further comprise a second actuator and a second sliding arm connected to the second actuator, for applying mechanical displacement to the biological sample in the bioreactor device in a second predetermined direction different from the first predetermined direction. The second predetermined direction can be perpendicular to the first predetermined direction. (See e.g., FIG. 11)

FIG. 11 depicts apparatus 108 according to an embodiment of the present invention wherein two sliding arms 103 and 104, guided by two sets of guiding rails, 101 and 102 respectively, are perpendicular from each other. In this embodiment, sliding arm 103 is actuated by actuator 105 and sliding arm 104 is actuated by actuator 106. The sliding arms 103 and 104 apply mechanical displacement to bioreactors 107 from two perpendicular directions.

Although FIG. 11 depicts an embodiment where two sliding arms of the load application unit form a 90 degree angle, embodiments wherein two or more sliding arms form angles other than 90 degrees are also within the scope of the present invention.

One or more sides of the bioreactor device can have a range of transparency properties that allows the biological sample to be visible to the user through at least one of unaided human vision and microscopy.

The base of the load application unit can be constructed from aluminum. The aluminum is preferably corrosion-resistant and/or aircraft-grade aluminum.

One or both of the sliding arm(s) and the guide rail(s) of the load application unit can be constructed from stainless steel.

The sliding arms of the load application unit and the actuators which actuate the sliding arms can provide for a range of motions. In one exemplary embodiment, the first and/or second sliding arm of the load application unit displaces the bioreactor device linearly. In another embodiment, the first sliding arm displaces the biological sample in the bioreactor device linearly in the first predetermined direction.

In one embodiment, the first and/or second actuator of the load application unit actuates linear displacement of the one or more of the sliding arms. In another embodiment, the first actuator actuates linear displacement of the first sliding arm in the first predetermined direction and the second actuator actuates linear displacement of the second sliding arm. The actuator(s) can be powered by a high-precision stepper motor.

In another aspect, the apparatus further comprises a control device configured to control the at least one of the first and the second actuator and/or at least one of the first and the second sliding arm. For example, such control can be effected through computer and one or more sensors.

In addition to the features described above, the apparatus disclosed herein can further comprise bearings configured to guide, in conjunction with the guiding rail, the displacement of the first sliding arm. For example, a ball bearing system connecting the sliding arm and the guiding rail can be used.

The apparatus provided by the present invention can further comprise one or more securing parts for securing the bioreactor devices onto the load application unit. The parts can include stop blocks and/or clamps. The stop blocks and/or clamps can be constructed from a polymer or a mixture of polymers which can include, for example, polyoxymethylene.

All combinations of the various elements described herein are within the scope of the invention.

The specific embodiments and examples described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

### Example of the Preferred Bioreactor System

The present disclosure is directed to a custom-made multi-part apparatus (the “bioreactor assembly”) that can be used to apply mechanical stimulation to biological samples, such as experimental scaffolds or biological grafts intended for tissue repair or regeneration. The bioreactor assembly can be used to grow tissue engineered grafts as well as condition allografts or autografts. The bioreactor assembly can also be used to research into fundamental questions such as the effects of bioreactor culture or biomechanical stimulation on stem cell differentiation as well as cell-matrix interactions.

The bioreactor assembly can be made of two separate components. A first component can be a bioreactor device that includes a casing that can house the biological sample, such as a scaffold. The second component can be a load application unit for applying mechanical stimulation to the biological samples housed in the bioreactor device.

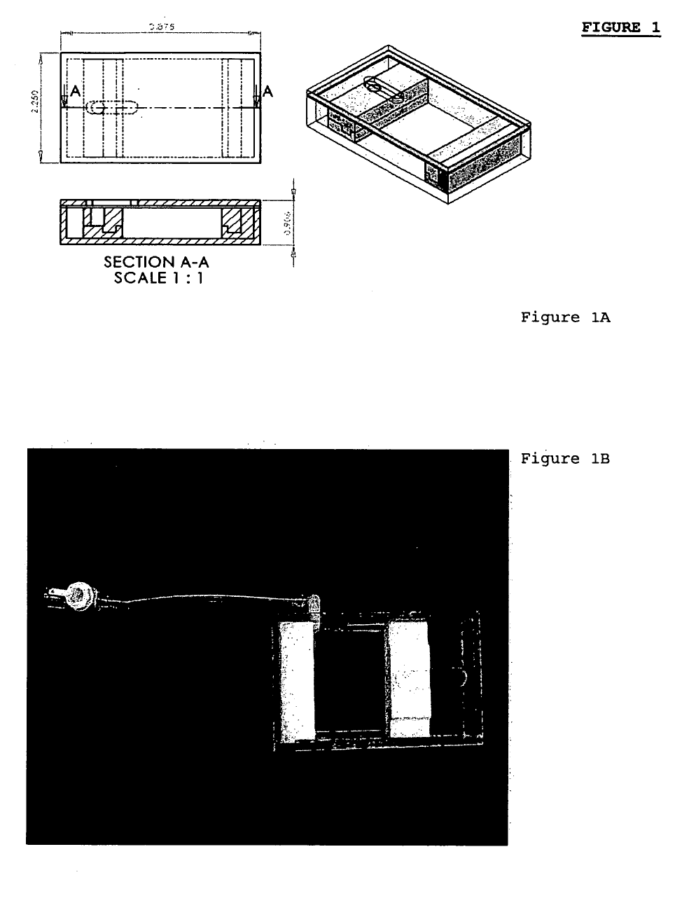

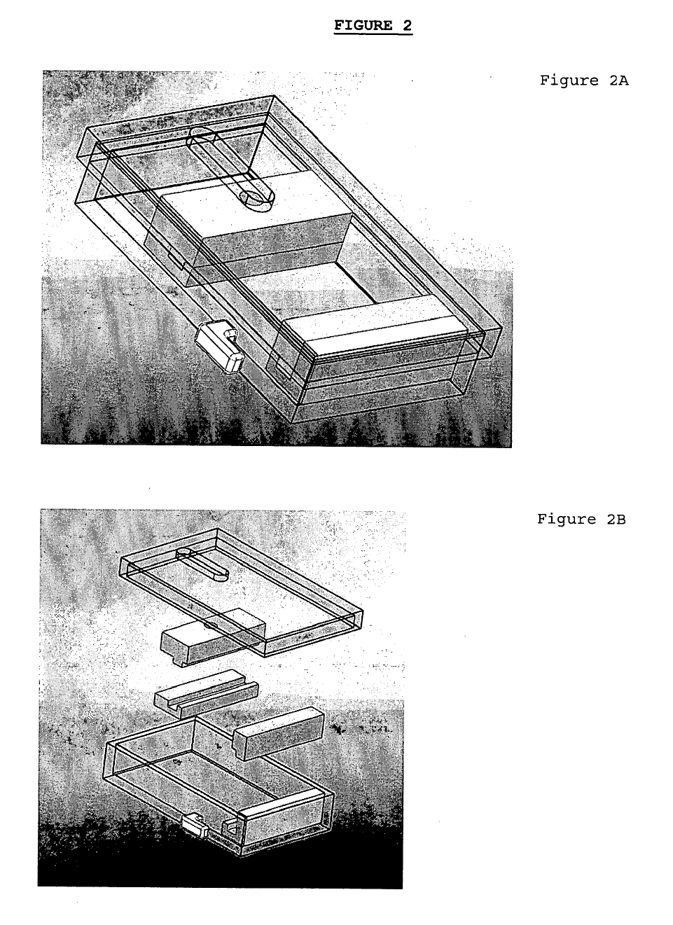

The bioreactor device provides the necessary environment for mechanical stimulation and/or mechanical conditioning of a cell/graft culture. The bioreactor device may be constructed from biocompatible, FDA-approved materials. Thus, the bioreactor device can be used with both tissue engineered scaffolds and biological grafts. Such a bioreactor device is illustrated in FIGS. 1 and 2. For example, the bioreactor device may have dimensions of H=0.906 inches, L=3.875 inches and W=2.25 inches.

The bioreactor device may be configured to meet three primary goals: 1) ease of use, 2) high sample-to-size ratio and 3) combine graft loading and culture. The bioreactor device parts, fabrication and assembly can be chosen and designed such that it can be fabricated reproducibly and economically. For example, the bioreactor device parts can be designed with nominal and commercially available dimensions. All or portions of the bioreactor device, such as the bioreactor device base, luer valves, and/or device tubing, may be designed to take advantage of commercial off-the-shelf components. Such a design criterion can result in relative ease in fabrication and assembly of the bioreactor device.

As shown in FIGS. 1 and 2, the bioreactor device may include, among other elements, a base and a cover for containing the biological sample therein. The base and cover maybe constructed from the same or different materials. Preferably, the base and cover are constructed from polymer materials. For example, the base may be formed from clear acrylic (polymethyl methacrylate, PMMA, Plexiglas) and the cover may be from polycarbonate (PC). Such a material selection allows a user approximately 360° C. of visibility of the biological specimen during loading of the bioreactor device. In addition, the transparent material selection for the bioreactor device allows for in situ image acquisition and biological specimen monitoring using either conventional or fluorescence microscopy.

The bioreactor device can also include a custom-made scaffold cutting board and/or bioreactor device clamp design. With such a configuration, the effort required in mounting a scaffold or graft can be minimized. Clamps as shown in FIGS. 1 and 2 can be used to secure the biological samples in place in the bioreactor device. The clamps may be made from a polymer material, such as polytetrafluoroethylene (PTFE, Teflon®). The material selection for the clamps can be chosen to allow smooth sliding of the clamps with respect to the base of the bioreactor device, while avoiding unwanted cell migration from the biological sample to the bioreactor device. Media can be contained within the bioreactor device by a cover over a gasket. Such as gasket may be fabricated from any gasket material known in the art, for example, silicone rubber.

Connection of the bioreactor device to the load application unit can be effected using a place-and-pin design. The bioreactor device can also be constructed with a relatively low profile characteristic with respect to the number of experimental samples it can hold. For example, the bioreactor device may be designed to have a 150% increase in space efficiency compared to other tensile loading designs. The bioreactor device may also be designed to have the ability to load biological samples, such as scaffolds, without unwanted deformation during the mounting process. The high sample-to-size ratio allows the bioreactor assembly to hold 40 samples per incubator shelf, and at least 20 samples per experimental strain variable. The bioreactor device also gives the user more degrees of observation and experimentation as compared to prior methodologies.

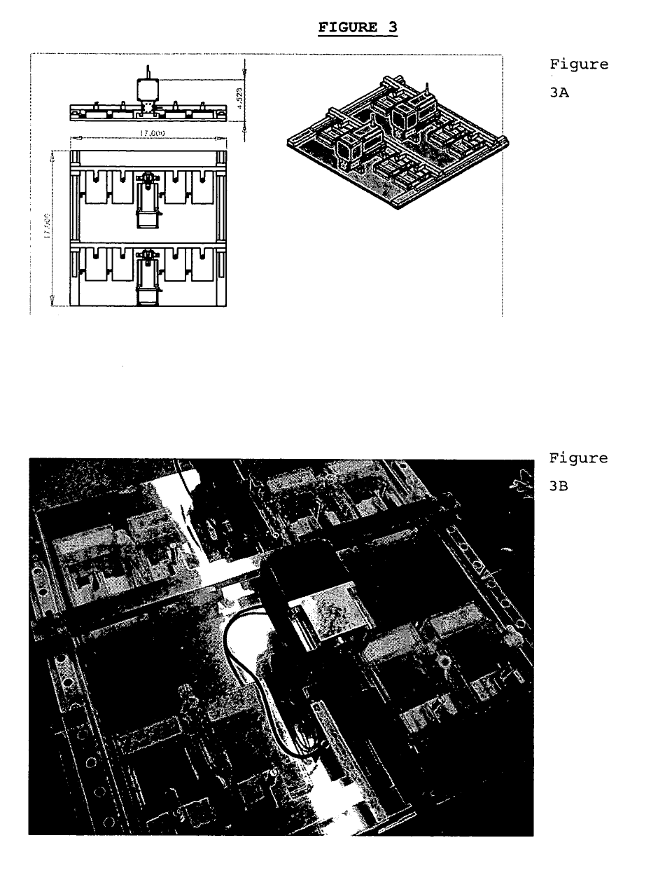

As shown in FIGS. 3A and 3B, the load application unit can include, among other elements, a base, a sliding arm, and an actuator. To accommodate a large range of mechanical deformation for application to the biological specimen in the bioreactor device, the load application unit base can be made from anti-corrosive, high strength materials. For example, the load application unit base may be formed using ultra-corrosion resistant, aircraft-grade aluminum. Such a material selection provides a lightweight, strong and easy to manufacture base that can withstand the humid environment of a biological incubator. For example, the bioreactor assembly may have dimensions of H=4.628 inches, L=17 inches and W=17 inches.

Mechanical loading of the bioreactor device is applied by the sliding arm of the load application unit. The sliding arm is connected to the actuator, which may be any type of precision actuator known in the art. For example, the actuator may be a linear actuator powered by a high-precision stepper motor. The sliding arm rides along a precision sliding system. The sliding system may be made from a metal, for example, stainless steel. Guide blocks slide over the rails by means of bearings, for example, ball bearings, to provide smooth and consistent movement.

The bioreactor device may be secured in place in the load application unit by stop blocks and fixture clamps. The stop block and fixture clamps may be constructed from a polymer material, for example, polyoxymethylene (POM, Delrin®). Such a polymer material selection provides a rigid stopping structure similar to metal material selection but without the risk of scratching the bioreactor device base. Furthermore, the fixture clamp is able to accommodate any discrepancies in bioreactor device dimensions by means of an adjustable head. Cover housing can be incorporated in order to protect each actuator section, as shown in FIG. 3B.

As shown in FIGS. 3A and 3B, for mechanical loading, two linear actuators can be powered by two stepper motors to provide force displacement of the biological specimens in the bioreactor devices. In order to keep the effects of temperature variations or magnetic fields on the incubator environment to a minimum, the load application unit motors and the bioreactor devices can be placed in different locations. Motor and actuator pairing can be accomplished by heavy duty sealed flexible coupling, which allows the respective motors to be located away from the bioreactor devices, such as outside an incubator. The motors can apply the mechanical displacement profile by means of a central controller (not shown).

Such a controller may tackle the form of, for example, a computer, network-enabled computer, or independent controller device. The controller can also be configured with the capability to drive multiple motors and respective actuators simultaneously. For example, the controller may drive up to four (4) separate stepper motors. Displacement profile(s) can be customized to an experimental protocol by varying the frequency of displacements, desired peak displacement, time of application and other similar profile parameters.

Furthermore, the bioreactor assembly as described herein may be configured to apply loads simultaneously in different profiles: tensile, compressive or a hybrid of both. Desired duty cycles can be set via the central controller. Because the load application unit can have two actuators, it has the versatility to run in a hybrid mode where part of the load application unit executes a tensile loading profile and another part of the load application unit executes a compressive loading profile. The bioreactor device can also be modified to be used as a compression bioreactor by removing the distance rods and turning the device 180° from its initial position. A force transducer can be placed in-line with the sliding portion of the bioreactor device in order to measure real-time force reactions.

The characteristics of the bioreactor assembly described herein enable flexibility in mounting either tissue-engineered scaffolds or biologically-derived soft-tissue grafts or scaffolds. The bioreactor device enable high-throughput as it is able to hold about five times more samples than most prior mechanical stimulation systems and has the capacity to hold up to forty samples using significantly less space. With minimal modifications, the bioreactor device can be rotated 180° to accommodate either compression or tensile loading profiles. The bioreactor assembly also allows for experiments to run simultaneously with different loading profiles and multiple chemical stimuli per individual loading profile. For example, in the bioreactor assembly illustrated in FIG. 3A, a total of 8 possible experiments can be running independently from each other. As the bioreactor device can be made from FDA-compliant components, the bioreactor device can provide a suitable environment for cellular sustainability and proliferation. Furthermore, the selection of industrial nominal sizes and parts make both the bioreactor device and load application unit extremely easy to fabricate and reproduce.

Furthermore, the foregoing description applies, in some cases, to examples generated in a laboratory, but these examples can be extended to production techniques. For example, where quantities and techniques apply to the laboratory examples, they should not be understood as limiting. Where noted above, disclosed dimensions and materials are for illustration purposes only and are not meant to be limiting.

It is, therefore, apparent that there is provided, in accordance with the present disclosure, a bioreactor assembly for mechanical stimulation of biological samples. Many alternatives, modification, and variations are enabled by the present disclosure. Features of the disclosed embodiments can be combined, rearranged, omitted, etc., within the scope of the invention to produce additional embodiments. Furthermore, certain features of the disclosed embodiments may sometimes be used to advantage without a corresponding use of other features. Accordingly, applicants intend to embrace all such alternatives, modifications, equivalents, and variations that are within the spirit and scope of the present disclosure.

Further aspects, features and non-limiting details are described in the following sections which are set forth to aid in an understanding of the subject matter.

The bioreactor assembly described herein (also the “Assembly”) has essentially two components, the bioreactor device (also the “Device”) and the load application unit (also the “Scaffold Loading System” or the “SLS”).

The bioreactor assembly was designed to address several research limitations present for the study of cell response. Primarily, these limitations arise from the absence of equipment to run large-scale stimulation tests, with the capability to incorporate and control mechanical and chemical stimulation parameters, as well as other experimental variables.

The bioreactor system described herein is a custom-made, multi-unit bioreactor system that can be used to apply mechanical stimulation to experimental scaffolds or biological grafts in order to study the behavioral response of cells seeded on the scaffold. Moreover, it can also be used to grow tissue engineered grafts and condition allografts or autografts.

### Example 1

### The Device

Referring to FIGS. 1 and 2, the Device provides the necessary environment for mechanical load application and cell culture. Designed with FDA-approved material, the Device is intended to be used with both tissue engineered scaffolds and biological grafts. Parts of the Devices are presently used widely in laboratories or medical procedures, or both. The Device components, functionality and future applications have been considered in both the design and fabrication processes in order to produce its current bioreactor capabilities. Its configuration was based on three primary goals: 1) ease of use, 2) high sample-to-size ratio and 3) combined loading and culture. Device parts, fabrication and assembly are chosen and designed such that it can be fabricated reproducibly and economically. Parts are in nominal and commercially available dimensions, with some parts (e.g. Device base, luer valves, tubing) available through third-party, custom-fabricating companies. This design criterion makes fabrication and assembly of the Device very approachable. Scaffold mounting is effortless with the aid of the custom-made scaffold cutting board and Device clamp design. Device connection and mounting to the SLS is straightforward and easy with the place-and-pin design.

Another advantageous characteristic of the Device is its low profile characteristics relative to the number of experimental samples it can hold. The current design results in a 150% increase in space efficiency compared to the previous tensile loading design, and the ability to load the scaffolds without the danger of deforming them during mounting. This high sample-to-size ratio allows the Assembly to hold 40 samples per incubator shelf, and at least 20 samples per experimental strain variable.

The Device also gives the user more degrees of observation and experimentation that most designs currently available, while being made from biocompatible materials compliant with FDA standards. The clear acrylic base and polycarbonate cover give the user about 360 degrees of visibility on the scaffold and cellular response. In addition, the Device's translucent casing design allows for in vitro scaffold image acquisition using conventional and fluorescence microscopy. Scaffold integrity and load transfer is achieved by Teflon® clamps. These Teflon® clamps allow for smooth sliding on the Device base while keeping the cells from proliferating on areas other than the scaffold, since cells do not attach on Teflon®. Media and any possible liquid spill mishaps are contained within the Device by a silicone rubber gasket.

A preferred embodiment is shown in FIGS. 1 and 2. The advantages of this design include:
1. Fabrication in the machine shop and third-party ordering of the base part is not required. Complete device can be fabricated in-house.
2. As compared to previous designs, gasket can be removed and sides can be added to the cover to improve airflow and decrease moisture build-up.
3. Screwing is not required for easier operation.
4. All device casing and cover is acrylic. This makes for laser cutting machinability.

However, due to the gasket being absent, leakage from the sides of the device is possible. On the other hand, this problem is minimal since pressure from handling and pressing on the cover is enough to prevent most leakage. However, depending on the elbow mounting, leaking may still occur. Therefore, it is preferable to seal the elbow with silicone on the inside before threading the elbow.

### Device Base

A main purpose of the base of the bioreactor device is to create an environment that is suitable for cell growth, and provide a platform to enable dynamic loading. It is also desirable that the base be durable and able to endure sterilization.

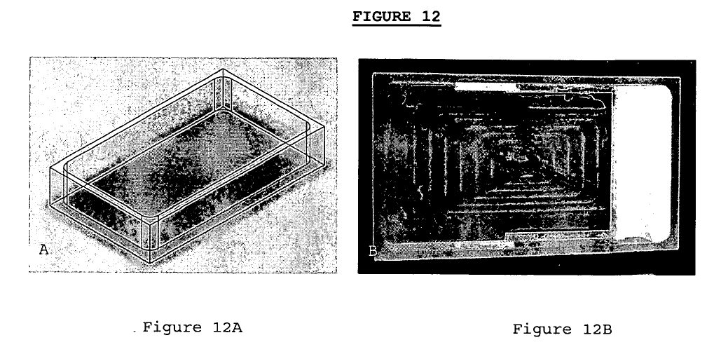

Therefore, in a preferred embodiment, the base of the Device is machined from a solid block rather than manufactured by using any acrylic solvent to weld together five pieces cut with a laser cutter. Because this base is milled from a solid block, the chance of leaking occurring through the seams in the bioreactor device is reduced or eliminated. FIG. 12A shows a CAD model of this single block Device base design. FIG. 12B shows a photograph of the single block Device base.

### Internal Teflon® Components

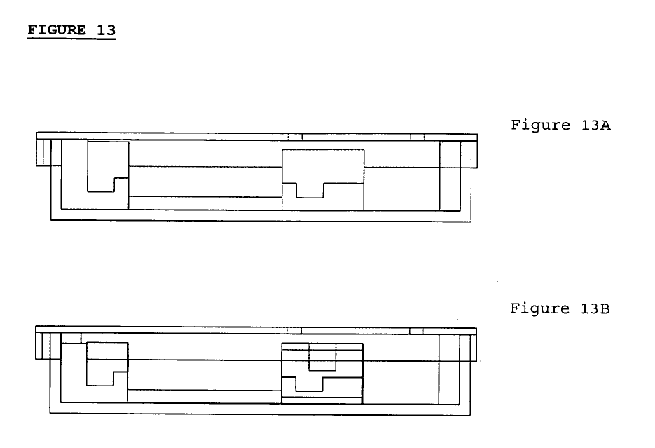

In a preferred embodiment, Teflon® pieces manufactured have a much tighter tolerance as compared to previous designs and do not leave any space between the walls of the device and the components. Also, the height of the Teflon® pieces was reduced from previous designs so that even if gaps occur or if capillary action is occurring, there is no path all the way to the top of the device. Removing this continuous pathway should reduce or prevent chances of leaking by capillary action. FIG. 13A shows the side view of the Device prior to the Teflon® pieces height adjustment. FIG. 13B shows the side view of the Device after this Teflon® pieces height adjustment.

### Device Cover

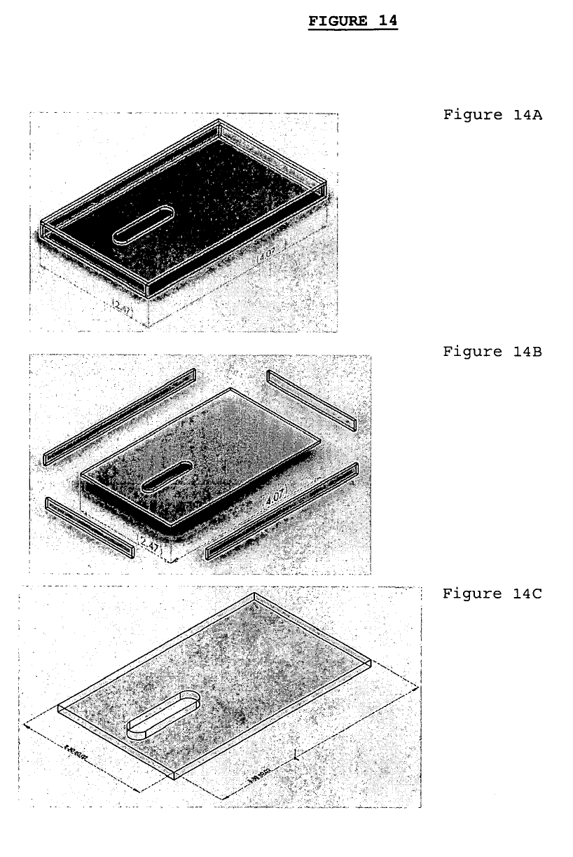

The Device cover in an exemplary embodiment of the present invention is shown in FIG. 14C. In this embodiment, the cover has a footprint the same size as the Device itself.

In a preferred embodiment, a design similar to a standard petri dish was employed for the Device cover. (See FIG. 14A) The footprint of this cover is larger than some of the prior designs, and the overhang allows the cover to be bumped or tilted without falling off. The assembly of the Device can be accomplished by using acrylic solvent to weld together five pieces, as shown in FIG. 14B. The interface between the base and the cover is tight enough that minor sloshing does not result in spills. The lack of gasket also prevents moisture from accumulating between the cover and the rim of the base, reducing or preventing chances of leakage due to condensation that may form on the cover of the device.

### Example 2

### The SLS

Large mechanical forces exerted on the scaffolds require that the SLS have a strong base to withstand deformation, while remaining lightweight so that most users can carry it. With this in mind, the SLS base, as well as any other custom-manufactured metal part from the SLS, is made from ultra-corrosion resistant, aircraft-grade aluminum. This material provides a lightweight, tough base that can withstand the humid environment of a biological incubator, while also being easy to manufacture. The Device applies mechanical load by a sliding arm that is connected to the actuator. This arm rides along a precision sliding system made from stainless steel. The guide blocks slide over the rails by means of ball bearings for a smooth and consistent movement. Device placement is secured by a Delrin® stop block and fixture clamps. Delrin® provides a rigid stopping structure like metal would without the risk of scratching the acrylic base, while the fixture clamp is able to accommodate any discrepancies in Device dimensions by means of an adjustable head. For further protection against fungi spores and other airborne pathogens, a SLS cover housing covers each actuator section (see FIG. 3B), with removable cover (not shown in picture) over each Device pair.

Two linear actuators, powered by two stepper motors, provide the force displacement. In order to keep the effects of temperature variations or magnetic fields on the incubator environment to a minimum, the SLS motor and the Assembly are placed in two different locations. Motor and actuator pairing is accomplished by a heavy duty sealed flexible coupling, which permits locating the motors outside the incubator. The stepper motors apply the mechanical displacement profile by means of a central controller, which has the capability to drive up to four (4) separate stepper motors. Displacement profile(s) can be customized to the experimental protocol by varying the frequency of displacement, desired peak displacement, time of application and other similar profile parameters.

Additional features are integrated in the design for different applications that may arise in the future. The Assembly has the capability to apply loads in three different profiles: tensile, compressive or hybrid. Because the Assembly is includes two stepper motors, it has the versatility to run in hybrid mode where part of the Assembly executes a tensile loading profile and another part of the Assembly a compressive profile. The Device can be modified to be used as a compression bioreactor by removing the distance rods and placing the device in a 180° orientation from tensile loading. In addition, a force transducer can be placed in-line with the sliding portion of the Device in order to measure real-time force reactions.

### SLS Plate

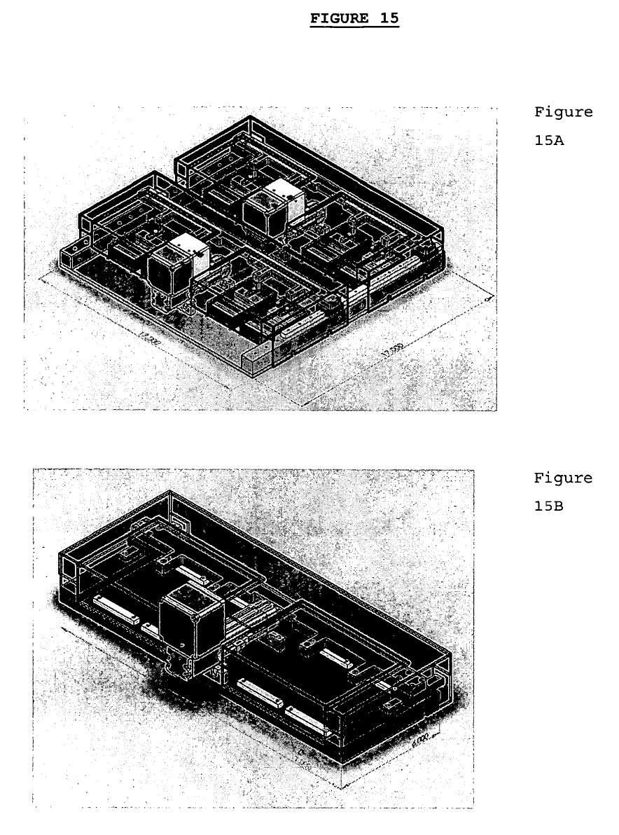

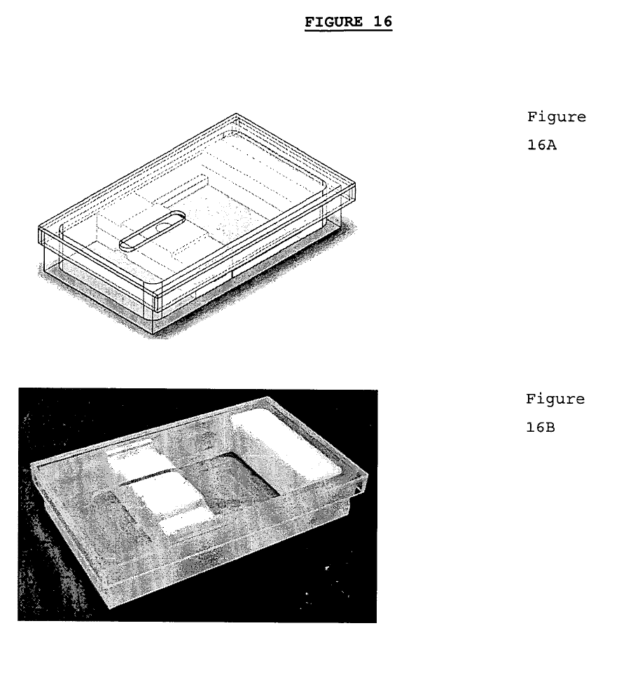

Two embodiments of a SLS plate are shown in FIGS. 15A and 15B.

In FIG. 15A, the plate measures 17×17 inches and the base plate and rail raiser bars are made from aluminum. The devices are held in place with a Delrin® bar fixed to the plate with stainless steel screws in the back, and by off center screws in the front. These screws function as follows: The head of the screw is not centered over its shaft, if one were to look along the shaft at the head while the screw is spinning the head would appear to wobble. When fitted inside a hexagonal nut this screw design can work as a fastening device. It functions just like the cam; the large part of the head presses up into the device and secure it, while the small part of the head move away and allow the device to be removed.

In FIG. 15B, the plate is less than half the size as that in FIG. 15A. This plate houses only four bio-reactors but the smaller plate is much easier to move in and out of an incubator and to maneuver. It is still possible to load more than four reactors by using more than one plate at once. This plate is made from Ultem®, a polyetherimide plastic. Ultem® is a relatively clear, amber colored material that has high strength, corrosion resistance to many solvents used in the laboratory, and high heat resistance. This material prevents corrosion from media spills, makes the plate easier to handle due to its lighter weight, and even allows for autoclave sterilization due to high temperature resistance. This design also uses Delrin® bars to hold in the bioreactor from both sides instead of just the back. This is to combat corrosion that was present in the non-stainless steel screws needed for the previous design.

### Example 3

### System Fabrication

### 3.1 Raw Materials

Raw materials are those materials which are bought in a predetermined size and brought to design dimensions with fabrication techniques. As a general rule, it is best to acquire the raw material with dimension(s) as close to those of the desired design dimension(s). Most of the raw materials described herein can be purchased through McMaster Carr (Elmhurst, Ill.).

When working with a raw material it is preferable to do the following:
1. Note any nicks from the supplier: In order to attain an accurate measure, these nicks should be shaved down before starting any fabrication on the piece.
2. Facing should be done on all faces of the piece: For a more precise measurement, target design dimensions should be taken after facing the piece. (Facing is a method used in machining that ensures that opposite sides are parallel and touching sides are perpendicular, thus making a perfect rectangle. It is a critical step for the accuracy of the dimensions.)

Other factors are preferably taken into account if the piece is to be used in a specific way. An example of the implementation of these factors is the system plate. Aluminum was chosen because of its lightweight properties and ease of fabrication. Further study showed that its Al 7075 alloy was the top choice because of its superior machinability, strength and corrosion resistance as well as its lightweight characteristic. This type of alloy is the same used in aircrafts because of these desirable characteristics.

### 3.1.1 Material Passivation

The SLS system is subjected to humid/wet environments since its main experimental location is inside a cell culture incubation system. In addition, its main component (the Device) is generally be filled with liquid, which might accidentally spill. Because of the ionic nature of the cell culture media, the system plate reacts with the media as pitting corrosion when both come in contact for a prolonged period (this does not happen with DI water, as previously tested).

Although Al 7075 is the main metallic component of the SLS, it is preferred to clean every piece of the SLS system prior to assembly. Failure to do so might allow foreign metals to remain stuck between parts and initiate a corrosive chemical reaction. For a greater measure of corrosion resistance, passivation the surface of the pieces is recommended (anodizing is also possible, but might be more expensive).

Before the actual passivation of the material, it is preferably that the piece be thoroughly cleaned. Only after doing so can the piece be subjected to the chemical reactions necessary for passivation. It is important to note that the proposed process takes approximately 9 days (Shih, 1992).

### A. Pre-treatment Process:

1. Thoroughly degrease/clean the piece with an alkaline cleaner (such as Alconox), rinse with warm distilled water to remove the detergent, and allow to air dry.
2. Fully immerse sample in 66° C. (150° F.) hexane for 15 minutes, rinse with distilled water, and allow to air-dry.
3. Remove surface particulates with an ultrasonic cleaner using acetone as the submerging liquid at 30° C. (85° F.). Again, rinse with distilled water, and allow to air-dry.
4. Remove residual organic reagents on the surface by repeating step 1.

### B. Deoxidizing Treatment:

1. Bathe washed sample in a chromate/HNO₃ bath (deoxidizer 17, Amchem) for 10 minutes at room temperature.
2. Bake sample in an oven at 100° C. (212° F.) for at least 24 hours.

### C. Passivation Treatment:

The passivation duration is determined by the amount of desired passivation. Because cell feeding is usually done every 2-4 days depending on the cell, and assuming that any liquid spilled during that time is cleaned, passivation time should take into account that the corrosive liquid only touches the metal surface for the 2-4 day time frame. With this in mind, it is suggested the sample be submerged in a 10 mM CeCl₃ solution for 7 days at room temperature. For the case of extreme increased exposure period (+20 days), passivation time can be done for 30 days with a solution of 10 mM CeCl₃.

Though chromate conversion coating is a more popular passivation method, it has been shown to produce hazardous by-products. This method has shown to provide equal or better corrosion resistance.

### 3.2 Fabrication Process

Make sure each piece can be fabricated from beginning to end without interruption. This ensures that the same reference point, tool surface characteristics, tool surface temperature and machining technique is used throughout the piece machining life, and decreases any inconsistencies in the piece dimensions. It should also be noted that in order to acquire a shine finish, the piece should be polished after machining.

### 3.2.1 Equipment Description

The SLS is fabricated from raw materials machined using a mill and a laser-cutter. Sawing and polishing are supplementary equipment that allow for easier machining to the design dimension.

### A. Horizontal and Vertical Band Saw

The horizontal band saw is used to cut raw materials to a more manageable dimension close to the design dimension. It is preferred that this cut be made with enough material left on the raw material to do a successful facing and final cut. This band saw type can be used for all materials, but is usually reserved for tougher materials such as steel and other hard metals, or any large (long) piece that needs to be cut down.

More versatile, the vertical band saw is used for closer cuts. Only plastics should be cut with this saw, and with the appropriate band saw blade, some soft metals like aluminum (depending on the type of aluminum, for example, Al 7075 should not be cut using the brand saw because of its toughness). It is best to accommodate the roller bearing on the blade track as close to the cutting face as possible.

### B. Bridgeport (by Hardinge) EZ Plus Vertical Mill

Before starting to mill the piece towards the design dimension, make sure to face the piece in order to assure the square geometry of the sides. It is important to note several factors that are preferably taken into consideration when milling:
1. Determine whether the piece can fit on the vice.
   a. For the SLS plate, the vice is removed and clamped directly onto the milling table. The plate is aligned such that the sides are parallel to the milling table to ensure a straight line of operation.
   b. For the sliding arm, complementing stage clamps should be improvised in order to deter the piece from vibrating.
2. Determine the adequate parameters for the tool type, tool velocity and feed rate in relation to the material to be milled. (“MEConsultant” can be used to calculate the correct operating parameters).
3. Identify all edges in relation to the static part of the vice clamp. Remember the side of the piece that was initially touching that part of the vice when facing the piece. This produces a better facing surface.

### C. Versa Laser 2.30

Made by Universal Laser Systems, the Versa Laser uses a 30 watt, far-infrared CO₂ laser beam capable of cutting through most plastics at 1 inch thick and engraves most metals. The laser-cutter proves to be extremely reliable in fabricating the Device and the SLS cover, as both are made from acrylic; a material superbly easy to work with using this equipment. All that has to be done in order to cut a piece is draw the desired geometry on Corel Draw and print it on the laser-cutter.

### 3.2.2 Parts Fabrication: Tips and Tricks

This section should be used as a guideline in the fabrication of the SLS. Several different set-ups were attempted before arriving to the most effective (which is presented here), but are by no means the only methods capable of arriving to the desired result.

It is preferred that the cutting tool (drill bit or end-mill) be thoroughly oiled at all times. This results in a cleaner finish, increased tool life, decreased temperature rise and vibrations. This process is time consuming and should be taken into consideration before starting the machining of the part.

It is also preferred to adequately clamp any oversized piece. Failure to adequately clamp an oversized piece can ultimately shatter the cutting tool and/or damage the piece's surface.

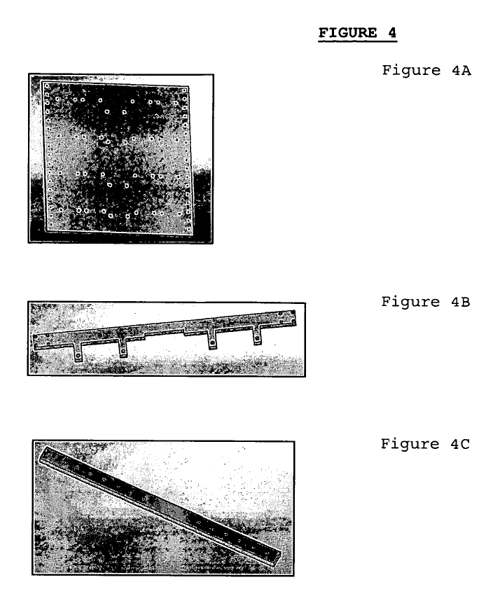

### A. SLS Plate (FIG. 4A)

Made from aircraft-grade, corrosion-resistant Al 7075, the plate exhibits excellent mechanical characteristics and machinability with an impressive light-weight feature. To machine the stock ordered from McMaster, the plate is first placed on the mill table by removing the vice, and clamping it down with the screw-down bumpers. After doing so, follow these guidelines:
1. Start to align the plate with right angles, clamping the angles if necessary. Finish aligning the plate by running the edge-finder along the x-axis of the plate. With the rubber hammer, align each corner as needed.
   A sheet of wood should be placed under the plate before aligning for the second time. This sacrificial wood is needed for the drilling of holes later.
2. Shave the necessary amount from two perpendicular edges to get to the design dimension. The plate needs to be re-aligned when it is moved shave the second side.
3. Write a “drill line” program to drill the holes on either side of the plate.
   a. Mill head forward/backward to drill the other side of the plate. Remember to re-zero that edge.
   b. From this point forward, remember where the front of the plate is; all other hole locations and pieces depend on this feature.
4. Drill all other holes either by writing a drill program (not recommended because of diverse hole placements) or drill each hole with their coordinates (tedious, but safe).
5. Finish by threading the holes, degreasing and polishing the surface.

It is preferable to keep the tool clean from shavings and always oil the piece surface during machining. Another aspect to have in mind is tool wear, as it can alter final piece dimensions. In order to minimize its impact on final dimensions, measure the actual tool dimensions with a caliper and use that for machining calculations.

### B. SLS Sliding Arm (FIG. 4B)

Fabrication of the arm is facilitated by a program that was written to machine the piece in a three-step process: milling starts on the left, followed by the right side and finishing by milling the center portion As with any new program, test it by running it without cutting the piece. Before running the program, make sure that the middle portion of the arm is clamped on the vice with supporting clamps on either side. Also, it is preferred that the supporting clamps do not bend the arm. The supports can come from a variety of pieces that are found in the machine shop, but all should be as close to being square or right angles as possible to minimize bending. Finally, make sure that the clamps are out of reach of the cutting tool. For the holes, make sure to always have the top surface orientation in mind, especially for the holes used to hide the screw heads.

### C. SLS Rail Spacer (FIG. 4C)

The Rail Spacer takes on two aspects of the Arm and Plate fabrication. Its oversized profile makes is preferable to be clamped like the Arm, and its hole pattern follows that of the Plate (i.e.: the same program for the Plate can be used here as well). As always, it is preferred to have in mind the orientation of the front part of the Rail Spacer and its association to the Plate.

All measurements and hole number can be found on the drawing of the respective piece.

### D. Device and Clamps

Device fabrication is easier since bulk fabrication is possible once a single piece is fabricated correctly, so care should be taken to fabricate the first piece as close to the design dimension as possible. For pieces that have extruding features (i.e.: top static clamp and top carriage clamp), a tolerance of −0.001″ should be strictly followed, as anything less results in scaffold slippage.

When working with Teflon®, clamping for should be just enough so that the piece does not move when pushed. This small clamping force ensures that the piece does not bow after the cutting tool begins removing material. Because Teflon® is very compliant, the cutting tool simply passes through the piece with minimal resistance.

Fabrication of the Device base and cover is done with the in-house laser-cutter, making fabrication of the Device extremely easy and repeatable.

### 3.3 Third Party Parts

Third party parts are those parts that come ready to assemble into the SLS with little or no modification. A detailed list of parts is shown in Table 1.

TABLE 1
Detailed Material List

| Item | McMaster # | Description | Purpose | Quantity |
|---|---|---|---|---|
| 1 | 9037K13 | 18″ × 18″ × ¼″ Corrosion-Resistant | Plate | 1 |
| | | Aircraft-Grade Aluminum | | |
| 2 | 6725K42 | 350 mm Length Rail All Stainless Steel | Guide Rail | 2 |
| 3 | 6725K4 | All Stainless Steel Mini Guide Block | Guide Block | 4 |
| | | 34 mm Length | | |
| 4 | 9083K146 | Super Corrosion Resistant SS | Moving Arm | 0.24 |
| | | (Type 316/316L) ½″ Thick, 2″ Wide, | | |
| | | 6′ Length | | |
| 5 | 97395A494 | Type 316 Stainless Steel Dowel Pin | Connecting Rod | 2 |
| | | ¼″ Diameter, 1½″ Length (pack of 5) | | |
| 6 | 92185A561 | Type 316 Stainless Stl Socket Head Cap | Arm/Act. Screw | 1 |
| | | Screw ¼″-28 Thread, ¾″ Length | | |
| | | (pack of 10) | | |
| 7 | 84865A31 | Low-Profile Clamp 8-32 × 11/32″ | Device Clamp | 16 |
| | | Thread Size 0.11″ Hex H × W 5/16″ | | |
| 8 | 92185A077 | Type 316 Stainless Stl Socket Head Cap | Stopper/feet Screw | 3 |
| | | Screw 2-56 Thread, ¼″ Length | | |
| | | (pack of 25) | | |
| 9 | 6426K84 | Heavy Duty Flexible Drive Shaft with | Motor Coupling | 2 |
| | | Ball Bearings, 19/32″ Shaft OD, | | |
| | | 4″ Length | | |
| 10 | 9055K341 | Aircraft grade A1 2½″ Thick, 3″ Wide | Actuator Adapter | 0.5 |
| | | 1″ Length | | |
| 11 | 9095K98 | High-Strength Stainless Steel, | Adapter Shaft | 0.5 |
| | | ¼″ Diameter | | |
| 12 | 5908K11 | 316 SS Ball Bearing for ¼″ ID | Adapter Bearings | 4 |
| | | ⅝″ OD, .196″ Width | | |
| 13 | 2457K16 | SS Universal Joint 1″ Joint Diameter | Adapter Coupler | 1 |
| | | 3⅜″ Overall Length | | |
| 14 | custom | Linear Motion Package | | 2 |
| 15 | custom | RSA12 Actuator | | |
| 16 | custom | Ball Nut, Low-Backlash | | |
| 17 | custom | Mounting Plates (×2) | | |
| 18 | custom | Limit Switch Sensor | | |
| 19 | custom | MRS232 Stepper Motor | | |
| 20 | custom | DS Stepper Drive | | |
| 21 | custom | Smart Hub Controller | | 1 |
| 22 | 5703T76 | Telecom Cord 14″ Modular | Controller Cables | 2 |
| | | Plugs, Black | | |
| 23 | custom | Acrylic Box | Device Base | 10 |
| 24 | 8735K45 | Teflon ® (⅝″ Thick, 2″ × 12″) | Static Clamp (Btm) | 0.5 |
| 25 | 9711K31 | Teflon ® (½″ Thick, 2″ × 12″) | Top Carriage | 0.5 |
| 26 | 9711K31 | Teflon ® (½″ Thick, 2″ × 12″) | Static Clamp (Top) | 0.5 |
| 27 | 9711K11 | Teflon ® (¼″ Thick, 2″ × 12″) | Btm Carriage | 1 |

1. ⅛″ #10-32, 90° Feeding Port Elbow (Ark-Plas Products, Inc.; www.ark-plas.com)
2. Ball Bearing Rail System (McMaster Carr; www.mcmaster.com)
3. Dowel Pins (McMaster Carr; www.mcmaster.com)
4. ⅛″ ID Tygon ® tubing (Cole-Parmer; www.coleparmer.com)
5. Linear Movement System and Controllers (Tolomatic, Inc.; www.tolomatic.com)
6. Device base (Riddout Plastics; www.ridoutplastics.com)

### Example 4

### System Assembly

Assembly is preferably done after all of the required materials are available. This allows the person assembling the system to calibrate and tweak the system all at the same time. Also, if the system is to be buffed or passivated, any of these two processes should be done before assembly takes place. Finally, in order to minimized corrosion and contamination, make sure that the system is completely washed from machining particulates and grease, and that no debris is left between two adjoining surfaces.

### 4.1 SLS Assembly

Finish the machined pieces by thoroughly cleaning and degreasing them, followed by any surface treatment(s). As part of the design goal, the SLS is easily put together following these guidelines:
1. Couple the linear slider/guide rail on top of the rail spacer and screw onto the SLS plate. The slider/guide rail (which is shorter than the SLS plate and rail spacer) should be on the front edge of the SLS plate. Do not fully tighten the screws.
2. Slide the ball-bearing linear sliders onto the rails and screw the SLS arm onto the sliders. Do not fully tighten the screws.
3. Correctly align the through-holes on the actuator supports with its placement on the SLS plate (the supports are held in place by 4 screws), and tightly screw the actuators onto the SLS plate.
4. Join the SLS arm with the actuator rod and tightly screw together.
5. Tighten but do not over-tighten the screws on the SLS arm. Over-tightening might lock the slider in place or alter its smooth movements.
6. Finally, slowly tighten the screws on the rail after correctly adjusting it to the actuator's movement. Adjustment is made by moving the SLS arm back and forth. This reorients the rail with the actuator's linear movement, and ensures smooth operation.

Before using the SLS System, make sure to make any final adjustments and corrections. It is important to note any jerky motion or “sticking” of the SLS arm as well as any part that rotates or shifts out of place as this could interfere with force transfer to the experimental specimen.

### 4.2 Device Assembly

Depending on the origin of the Device (whether acquired from a third-party entity or fabricated in-house) minimal assembly is needed. For either case, all Teflon® parts are fabricated in the machine shop and the Device cover with the laser-cutter.

Freely apply the silicone adhesive on the bottom static clamp, and slowly apply pressure on the clamp. This ensures that no air bubbles are left between the clamp and the device base

### 4.2.1 Third Party Acquisition

By acquiring the Device from a third party entity, previous results have given a tolerance of ±0.034″ which, with the current Device clamping mechanism, is not acceptable since it complicates the System by adding another feature that needs calibration. A tolerance of ±0.01″ or less is deemed acceptable for the current application.

The only assembly step for this method is adhesion of the bottom static clamp. Apply the silicone RTV adhesive on both surfaces of the piece that come in contact with the Device base.

### 4.2.2 In-House Fabrication

After laser-cutting the Device pieces, proceed to assemble the Device by adhering the pieces with acrylic weld (Weld-on #3; McMaster Carr). For correct assembly, the sides are preferably parallel to each other and perpendicular to the bottom. This can be achieved by creating a mold to drop each side in and then applying the acrylic weld, or by using right-angled and parallel straightedges to correctly orient the pieces individually. Either of these processes can be used to assemble the base and the cover. Finally, proceed to place the bottom static clamp as described above.

In both methods, addition of the 90° feeding port elbow is done after placing the bottom static clamp. It is preferable to do so afterwards in order to err on the side of caution, in case the bottom static clamp is fabricated thicker than intended, over-lapping the drilled hole. Although the elbow has a #10-32 thread, it is best to tap the hole with a drill bit one size smaller than suggested in order to attain a tighter fit. Also, over-tighten the elbow can cause the hole to crack when placed in the incubator due to stress relaxation. Covering the threads with the RTV silicone prior to screwing the elbow onto the Device might help prevent any leaking problems.

### Example 5

### Alternative Designs

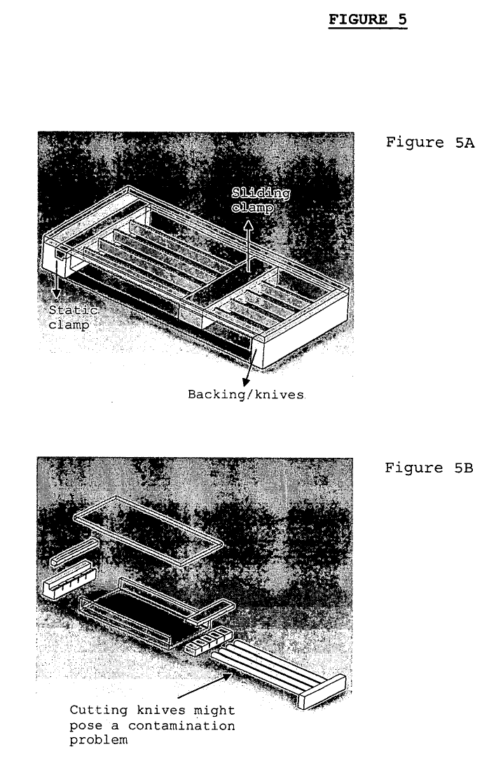

Several iterations of the Device were proposed before arriving at the version described supra. A number of alternative designs proposed are illustrated in FIGS. 5-9 and the summarized below and are also within the scope of the present invention.

5.1 Device 2i.1 (FIGS. 5A and 5B)

Device 2i.1 comprises polytetrafluoroethylene (PTFE) clamps, backing and slider.

The transparent material considered for the bioreactor device casing include: a) glass, which is relatively frictionless, clear, autoclavable and brittle, but hard to machine; b) polycarbonate which is biocompatible, clear, strong, easy to machine. However, polycarbonate looses optical properties when machined and is not autoclaveable; c) acrylic which is clear and easy to machine but not autoclavable or biocompatible.

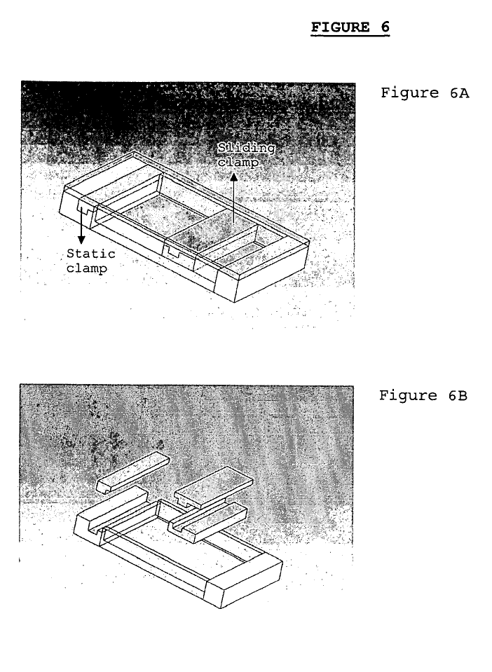

Further modifications considered include:
1. Removing or diminish crevices in the clamps which may promote cell residue formation;
2. Removing any material that is not used throughout the entire assay including the cutting mechanism;
3. Designing strain rods to come from the top or sides of the device rather than cross backing and connecting to the sliding clamp, moving in and out, which would have part of the connecting rod entering and exiting the closed system, increasing contamination probability; and
4. Generally minimizing all non-flat surface presence, one time use parts and media interaction.
   5.2 Device 2i.2 (FIGS. 6A and 68)

Device 2i.2 is designed with the same materials as the previous version.

Device 2i.2 is more compliant to established mesh cutting dimensions (5×6 cm) for 5 meshes (1×6 cm each), with some deviations (5×6.7 cm) due to manufacturing process limitations.

Also, gauge length of 4.14 cm (manufactured) vs. 4 cm (current) is even more precise since it is more stringent for mesh characterization.

Compared to Device 2i.1, Device 2i.2 has increased dimensions for easier manufacturing and screwing applications. Also, a total of 0.5″ (¼+⅛+⅛) on each clamp is used for clamping to allow for the established mesh length of 6 cm (real→6.667 cm). In addition, a 1⅜″ distance (4.14 cm) is left as the gauge length, for a width-to-length ratio of approximately 1:4. Finally, the space left accounts for a strain of 25%.

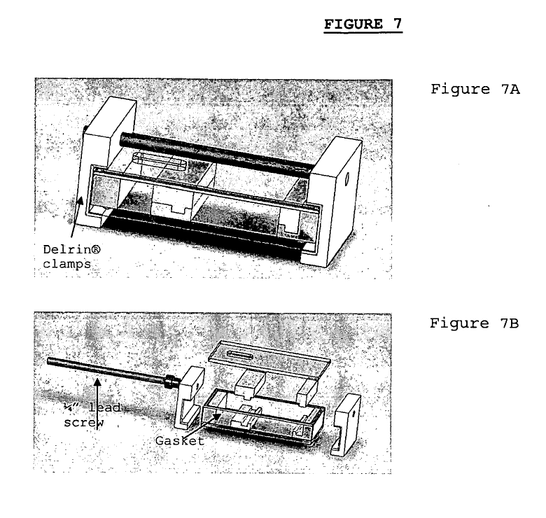

Further modifications considered include:
1. Incorporating the ends and the cover inside the bottom and sides to minimize cracks and associated leakage;
2. Leaving the mesh height the same, but make all other parts higher . . . ˜0.25″ to increase distance from the media and the top;
3. Drilling holes on the bottom slider or create ditches on the bottom tray to reduce or eliminate media void on the side contrary to direction of motion created by the slider to bottom/side contact; and
4. Incorporating silicone gaskets on the top to reduce or eliminate leakage through the top.
   5.2 Device 2i.3 (FIGS. 7A and 78)

Compared to previous versions of the Device, Device 2i.3 includes the following modifications: a) incorporating rubber gaskets to prevent cover leakage; b) fabrication of clamps for cover clamps and lead screw placement; c) using Delrin® as cover clamping material; d) elimination of leakage from the side walls; e) introduction of static phase.

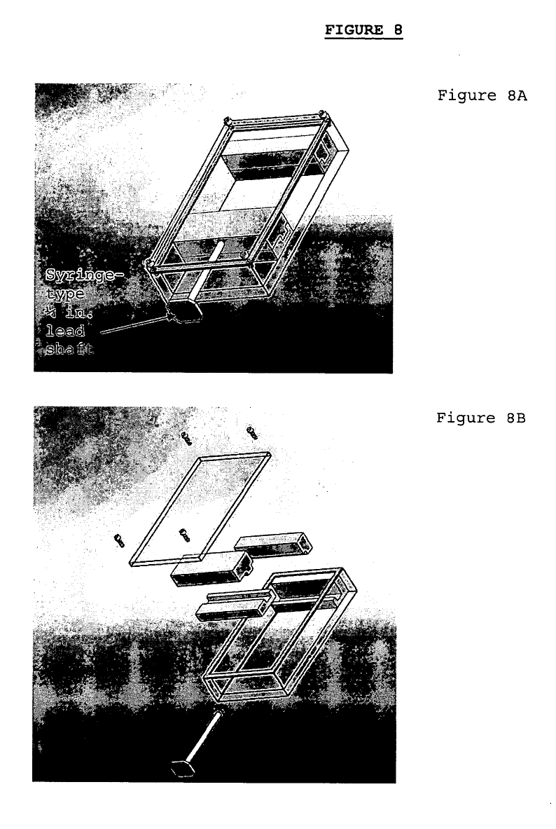

Further modifications considered include:
1. Incorporating design for easier seeding as scaffold seeding using the current design is harder than intended. Removal of the cover requires removal of the complete clamping assembly, which might stretch the scaffold (applying initial pre-straining) when disconnecting carriage from lead screw; and
2. Reducing the number of parts as the addition of clamping/lead-screw assembly increases complexity, which leads to greater routes for failure and/or contamination. Also, more parts extend to greater precision requirements in order to maintain controlled system strain and rigidity, which by current manufacturing technology, significantly increases production time and cost.
   5.3 Device 2i.4 (FIGS. 8A and 88)

Compared to previous versions of the Device, Device 2i.4 includes the following modifications: a) addition of flat-head screws to the clamps to ensure a secure grip and seal; b) removal of Delrin® grips and overhead lead screw assembly; c) use of a syringe-style attachment on the carriage for inline strain application; and d) ensuring enclosure of the internal chamber of the bioreactor device.

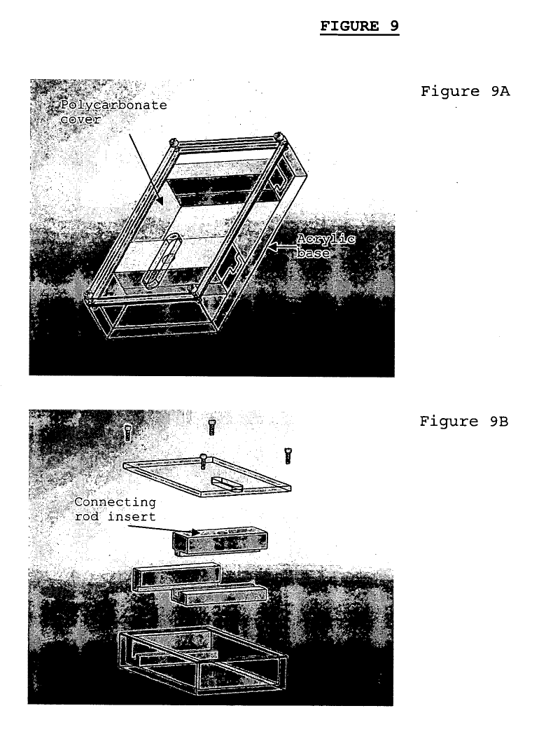

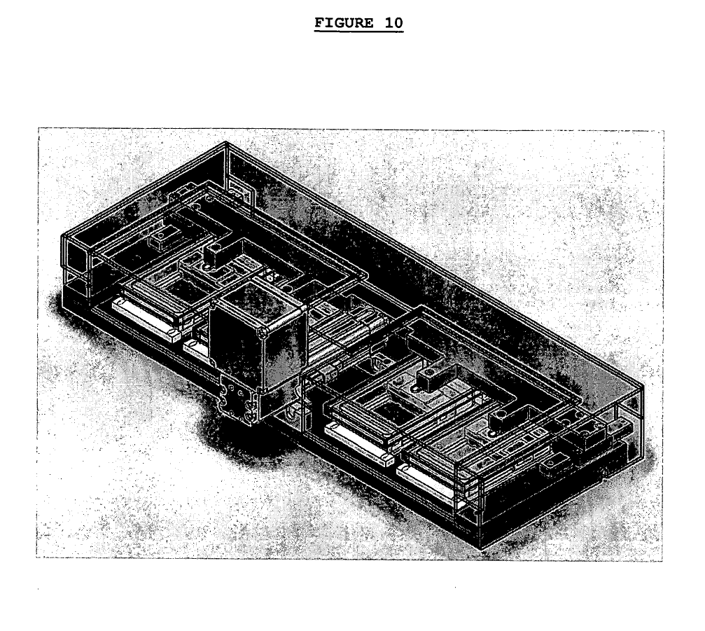

Further modifications considered include:
1. Improving or replacing syringe design as currently the proper sealing of the syringe is difficult to determine without adequate (long-time) testing, its joint can leak, causing a depletion of media and cell death, and its in and out motion introduces a environment-exposed orifice, promoting bioreactor contamination. In addition, syringe fabrication requires precise fabrication and several components, increasing production cost.
2. Adjusting gasket thickness as current gasket thickness allowed for clamp loosening from scaffold pulling resistive force. The gasket used was ⅛″ thick, giving more cover clearance for the clamps, allowing for clamp loosening and significant vertical displacement.
   5.4 Device 2i.5 (FIGS. 9A and 95B)

For Device 2i.5, use of an overhead approach is reconsidered, since strain application can occur by straining system. Compared to previous versions of the Device, Device 2i.5 includes the following modifications: a) removal of syringe-type lead shaft and reintroduction of connecting rod on carriage; b) pushing cover slit closer to edge to ensure effective usage of space and allowing for slit covering by strain system arm; c) custom fabricating base made from acrylic treated for improved resistance to UV degradation and scratching; and d) change gasket thickness from ⅛ inches to 1/16 inches or 1/32 inches.

Further modifications considered include:
1. Using alternative cover locking as acrylic base might not handle threading process; and
2. Ensuring proper sealing of slit, although previous model showed no contamination.

### REFERENCES

1. U.S. Pat. No. 6,432,712, issued Aug. 13, 2002 to Lloyd Wolfinbarger, Jr.
2. U.S. Pat. No. 7,179,287, issued Feb. 20, 2007 to Lloyd Wolfinbarger, Jr.
3. U.S. Pat. No. 7,348,175, issued Mar. 25, 2008 to Kent Vilendrer, et al.
4. Altman, Gregory H., et al. (2002) “Advanced Bioreactor with Controlled Application of Multi-Dimensional Strain.” J. Biomech. Eng. 124(6):742-749.
5. Screen, H. R. C. (2008) “Investigating Load Relaxation Mechanics In Tendon” Journal of the Mechanical Behavior of Biomedical Materials 1(1):51-58.
6. Screen, H. R. C., et al. (2003) “Development Of A Technique To Determine Strains In Tendons Using The Cell Nuclei” Biorheology 140 (2003)361-368.
7. Shih, H. and Mansfeld, F., “Passivation in Rare Earth Metal Cholrides—A New Conversion Coating Process for Aluminum Alloys,” New Methods for Corrosion Testing of Aluminum Alloys, ASTM STP 1134, V. S. Agarwala and G. M. Ugiansky, Eds., American Society for Testing and Materials, Philadelphia 1992, pp. 180-195.

1. An apparatus for applying mechanical loading to a biological sample, said apparatus comprising:
a bioreactor device configured to house the biological sample, and
a load application unit for applying mechanical loading to the biological sample housed in the bioreactor device, said load application unit including:
a) a first sliding arm for applying mechanical displacement to the biological sample in the bioreactor device in a first predetermined direction; and
b) a guide rail coupled to the first sliding arm to limit movement of the first sliding arm such that the first sliding arm moves only in the first predetermined direction when displaced.

2. The apparatus of claim 1, wherein the bioreactor device comprises:
c) a first securing device for securing one end of the biological sample; and
d) a second securing device for securing another end of the biological sample opposite to the first end;
wherein the load application unit further comprises:
e) an insert piece to couple the first sliding arm to the first securing device or the second securing device,
and wherein the first predetermined direction in which the sliding arm is displaced is perpendicular to a longitudinal axis of the insert piece.

3. The apparatus of claim 1, wherein the bioreactor device further comprises:
f) a static clamp for securing one end of the biological sample; and
g) a sliding clamp for securing another end of the biological sample opposite to the first end, said sliding clamp being configured for movement in said first predetermined direction.

4. The apparatus of claim 1, wherein one or more sides of the bioreactor device has transparency property that allows the biological sample to be visible to the user through at least one of unaided human vision and microscopy.

5. The apparatus of claim 1, wherein the load application unit further comprises a first actuator connected to the first sliding arm for actuating the mechanical displacement of the sliding arm in the first predetermined direction.

6. The apparatus of claim 5, wherein the load application unit further comprises a second actuator and a second sliding arm connected to the second actuator, for applying mechanical displacement to the biological sample in the bioreactor device in a second predetermined direction different from the first predetermined direction.

7. The apparatus of claim 6, wherein the first actuator actuates linear displacement of the first sliding arm in the first predetermined direction and the second actuator actuates linear displacement of the second sliding arm.

8. The apparatus of claim 1, wherein the first sliding arm displaces the biological sample in the bioreactor device linearly in the first predetermined direction.

9. The apparatus of claim 5, further comprising a control device configured to control at least one of the first actuator and the first sliding arm.

10. The apparatus of claim 1, further comprising bearings configured to guide, in conjunction with the guiding rail, the displacement of the first sliding arm.

11. The apparatus of claim 1, further comprising one or more securing parts for securing the bioreactor device onto the load application unit.

12. The apparatus of claim 2, wherein the load application unit further comprises a first actuator connected to the first sliding arm for actuating the mechanical displacement of the sliding arm in the first predetermined direction.

13. The apparatus of claim 12, wherein the load application unit further comprises a second actuator and a second sliding arm connected to the second actuator, for applying mechanical displacement to the biological sample in the bioreactor device in a second predetermined direction different from the first predetermined direction.

14. The apparatus of claim 13, wherein the first actuator actuates linear displacement of the first sliding arm in the first predetermined direction and the second actuator actuates linear displacement of the second sliding arm.

15. The apparatus of claim 3, wherein the load application unit further comprises a first actuator connected to the first sliding arm for actuating the mechanical displacement of the sliding arm in the first predetermined direction.

16. The apparatus of claim 15, wherein the load application unit further comprises a second actuator and a second sliding arm connected to the second actuator, for applying mechanical displacement to the biological sample in the bioreactor device in a second predetermined direction different from the first predetermined direction.

17. The apparatus of claim 16, wherein the first actuator actuates linear displacement of the first sliding arm in the first predetermined direction and the second actuator actuates linear displacement of the second sliding arm.

18. The apparatus of claim 4, wherein the load application unit further comprises a second actuator and a second sliding arm connected to the second actuator, for applying mechanical displacement to the biological sample in the bioreactor device in a second predetermined direction different from the first predetermined direction.

19. The apparatus of claim 18, wherein the first actuator actuates linear displacement of the first sliding arm in the first predetermined direction and the second actuator actuates linear displacement of the second sliding arm.

20. The apparatus of claim 19, wherein the load application unit further comprises a first actuator connected to the first sliding arm for actuating the mechanical displacement of the sliding arm in the first predetermined direction.
